(19) United States
(12) Patent Application Publication
(10) Pub. No.: US 2010/0241163 A1
(43) Pub. Date: Sep. 23, 2010

(54) Aortic Dissection Treatment System and Method of Use

(75) Inventors: Josiah Wilcox, Santa Rosa, CA (US); Julie Trudel, Santa Rosa, CA (US); James Machek, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/409,272

(22) Filed: Mar. 23, 2009

(51) Int. Cl. A61B 17/03

(52) U.S. Cl. 606/214

(57) ABSTRACT

An aortic dissection treatment system and method of use including a treatment system for an aortic dissection having a catheter defining a suction lumen and a sealant lumen; a flexible cup defining an isolation region within the flexible cup, the isolation region being in communication with the suction lumen and the sealant lumen; and a sealant fluid deliverable to the aortic dissection through the sealant lumen and the isolation region. The flexible cup is sized to fit over at least one end of the aortic dissection and the suction lumen applies suction to maintain the flexible cup over the at least one end of the aortic dissection.

### TECHNICAL FIELD

The technical field of this disclosure is medical treatment devices, particularly, an aortic dissection treatment system and method of use.

### BACKGROUND

Aortic dissection occurs when the intimal layer of the aorta tears, so that blood passes through the tear into the medial layer. Blood pressure within the aortic dissection can promote further tearing to increase the length of the initial aortic dissection, creating a false lumen under an intimal flap. In some cases, the false lumen is a through lumen with blood flowing through the false lumen in addition to the true lumen. In other cases, the false lumen is a partial lumen with the intimal flap forming a pocket in the aortic wall. Blood under pressure can increase the size of the aortic dissection and may force the intimal flap into the true lumen, reducing the cross-section of the true lumen of the aorta for blood flow. Aortic dissections can occur in the ascending aorta and/or aortic arch, classified as Type A, or in the descending aorta, classified as Type B. An aortic dissection associated with an aortic aneurysm can increase the chance of rupture of the aortic aneurysm due to the reduced strength of the aortic wall. Aortic dissection can be caused by aortic diseases, such as aortic dilation, aortic aneurysm, or Marfan syndrome, or by traumatic aortic injuries, which often occur in the thoracic aortas of relatively young persons, a population which is prone to accidents.

Currently, aortic dissections are repaired with open surgery or minimally invasive procedures. In open surgery, the diseased vessel segment is retracted and any entry to the false lumen from the true lumen is closed. While open surgery was and is an effective surgical technique in light of the high risk associated with a fatal aortic rupture, the open surgical technique suffers from a number of disadvantages. It is complex, requires a long hospital stay, requires a long recovery time, and has a high mortality rate. Open surgery can also be used to place a stent graft across the diseased vessel segment as in minimally invasive procedures, but stent grafts have their own disadvantages.

In less or minimally invasive procedures, a tubular endoluminal prosthesis that provides a tubular graft for blood flow while excluding blood flow to the aortic dissection site is introduced into the blood vessel using a catheter. The tubular endoluminal prosthesis is introduced in a small diameter compressed configuration and expanded at the aortic dissection. Often referred to as stent grafts, these tubular endoluminal prostheses are used to secure tubular graft material held open in a sealing engagement with the vessel wall by one or more stents as a support structure. Although stent grafts provide a short term solution, one disadvantage of stent grafts is that the stent graft maintains a fixed geometry, even though the vasculature of the patient may change over time. This problem is particularly acute in treatment of traumatic aortic injuries in young persons, whose aortas expand as they age. The radial expansion of present stent grafts is limited by the diameter of the graft material attached to the stent, typically being limited to ten to twenty percent oversizing of the original diameter of the stent graft as deployed in the vessel. This is often insufficient to maintain the stent graft seal with a growing aorta. When the stent graft diameter becomes too small compared to the surrounding vessel, the stent graft may leak and may even migrate from the deployment site to an undesirable location. Although the aorta may have healed from the trauma, risky and expensive surgery may be required to remove the now undersized stent.

One experimental approach to sealing aortic dissections has been published by Tanaka, et al. in The Annals of Thoracic Surgery, Volume 68, Issue 4, October 1999, Pages 1308-1312. In an animal study of surgical treatment of acute aortic dissection, the sealant AdvaSeal from Ethicon, Inc., was applied to the false cavity of surgically created acute descending aortic dissections for reinforcing and fusing the dissected layers and was also applied to the suture line. The experiment found the sealant was advantageous due to its effectiveness on wet tissue and its adhesiveness, allowing good hemostasis and closure of the false lumen. Unfortunately, the experimental approach requires that the clinician have direct access to the aortic dissection for application of the sealant, so expensive and risky open surgery is required.

It would be desirable to overcome the above disadvantages.

### SUMMARY OF THE INVENTION

One aspect according to the present invention provides a treatment system for an aortic dissection including a catheter defining a suction lumen and a sealant lumen; a flexible cup defining an isolation region within the flexible cup, the isolation region being in communication with the suction lumen and the sealant lumen; and a sealant fluid deliverable to the aortic dissection through the sealant lumen and the isolation region. The flexible cup is sized to fit over at least one end of the aortic dissection and the suction lumen applies suction to maintain the flexible cup over the at least one end of the aortic dissection.

Another aspect according to the present invention provides a treatment system for an aortic dissection in a vessel including a catheter defining an inflation lumen and a sealant lumen; a first balloon attached to the catheter, the first balloon defining a first perfusion opening and being in communication with the inflation lumen; a second balloon attached to the catheter, the second balloon defining a second perfusion opening and being in communication with the inflation lumen; a perfusion body connecting the first perfusion opening and the second perfusion opening, with adjoining exterior surfaces of the first balloon, the second balloon, and the perfusion body defining an isolation region when the first balloon and the second balloon are inflated in the vessel, the isolation region being in communication with the sealant lumen; and a sealant fluid deliverable to the aortic dissection through the sealant lumen and isolation region. The first balloon and the second balloon are separated by a distance along the catheter, the distance being selected to accommodate at least one end of the aortic dissection in the isolation region.

Another aspect according to the present invention provides a method of treatment for an aortic dissection in a vessel, the method including imaging the aortic dissection to locate an entrance; isolating the entrance from blood flow through the vessel without blocking the blood flow through the vessel; and applying sealant fluid to the entrance.

The foregoing and other features and advantages will become further apparent from the following detailed description, read in conjunction with the accompanying drawings. The detailed description and drawings are merely illustrative.

### BRIEF DESCRIPTION OF THE DRAWINGS

FIGS. 1A-1C are schematic side, top, and bottom views of a dissection treatment device;

FIGS. 2A-2C are schematic cross sectional side views showing progressive steps of covering a dissection;

FIGS. 3A-3C are schematic cross sectional side views showing progressive steps of filling a dissection;

FIG. 4 is a schematic side view of another embodiment of a dissection treatment device;

FIGS. 5A-5C are schematic side, bottom, and end views of a dissection treatment device;

FIGS. 6A-6C are schematic cross sectional side views showing progressive steps of covering a dissection;

FIGS. 7A-7C are schematic cross sectional side views showing progressive steps of filling a dissection; and

FIG. 8 is a flowchart of a method of dissection treatment.

### DETAILED DESCRIPTION

Embodiments according to the invention will now be described by reference to the figures wherein like numbers refer to like structures. The terms “distal” and “proximal” for the dissection treatment device are used herein with reference to the treating clinician during the use of the dissection treatment device: “distal” indicates a portion of the dissection treatment device distant from, or a direction away from the clinician and “proximal” indicates a portion of the dissection treatment device near to, or a direction towards the clinician. The terms “distal” and “proximal” for the aortic dissection are used herein with reference to the direction of blood flow from the patient's heart to and past the aortic dissection: “proximal” indicates a portion of the aortic dissection nearest the heart according to the blood flow path from the heart to the aortic dissection, “distal” indicates a portion of the aortic dissection distant from heart according to blood flow path, or the end opposite the proximal end. The distal end of the dissection treatment device can be at the proximal or distal of the aortic dissection during treatment depending on the location of the aortic dissection and the approach path through the vasculature.

The devices and methods are described below in terms of being used to treat aortic dissections, those skilled in the art will appreciate that the devices could be used to treat dissections in other vessels as well.

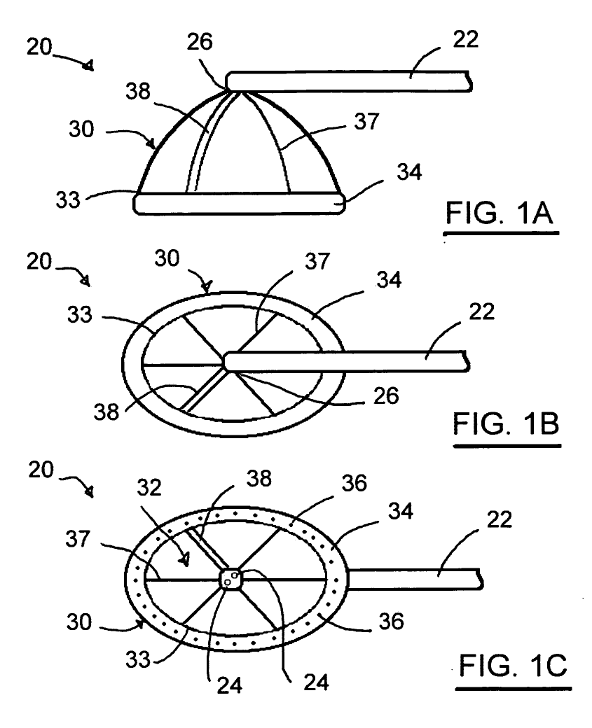

FIGS. 1A-1C are schematic side, top, and bottom views of a dissection treatment device. In this embodiment, a flexible cup isolates at least a portion of an aortic dissection. A sealant fluid is delivered to the aortic dissection at the isolated portion.

The dissection treatment device 20 includes a catheter 22 and a flexible cup 30. The catheter 22 defines a suction lumen (not shown) and one or more sealant lumens 24 through the length of the catheter 22. The flexible cup 30 defines an isolation region 32 within the flexible cup 30, with the isolation region 32 being in communication with the sealant lumen 24. Sealant fluid is deliverable to the aortic dissection through the sealant lumen 24 and isolation region 32. The flexible cup 30 is sized to fit over at least one end of the aortic dissection and the suction lumen applies suction to maintain the flexible cup 30 over the aortic dissection. In one embodiment, the flexible cup 30 is maintained over one end of the aortic dissection. In another embodiment, the flexible cup 30 is maintained over both ends of the aortic dissection. In operation, the distal end of the dissection treatment device 20 is delivered to the aortic dissection through a delivery catheter in a rolled and/or folded configuration. The flexible cup 30 unfurls to the illustrated configuration on exiting the delivery catheter. The flexible cup 30 is retracted into the delivery catheter after the aortic dissection has been sealed.

The flexible cup 30 can include ribs 37 disposed around the flexible cup between the catheter attachment 26 and the circumferential edge 33 of the flexible cup 30. The ribs 37 shape the flexible cup 30 during deployment and provide the desired flexibility to the flexible cup 30. The flexible cup 30 of this example includes a suction ring 34 around a circumferential edge 33 of the body of the flexible cup 30. The suction ring 34 holds the flexible cup 30 to the vessel wall through suction on the suction ports 36. The suction ring 34 can communicate with the suction lumen through one or more suction ribs 38. The size, number, and placement of the suction ports 36 on the suction ring 34 can be selected to provide the desired force to hold the flexible cup 30 against the vessel wall. Further, different suction ports 36 can be fed by different suction lumens to assure that the suction ring 34 remains anchored to the vessel wall even if some of the suction ports are not well seated on the vessel wall and leak. The flexible cup 30 can optionally include light sources, fiber optic cables connected to external light sources, and/or spray heads connected the sealant lumen as desired for the selected sealant fluid and treatment method.

The catheter 22 can be made of any flexible biocompatible material normally used for catheters. For example, the catheter 22 can be made of polymers such as polyurethane, polyethylene, polyether block amide (PEBAX), nylon, composites, or any combination of the above, or the like. The catheter 22 is long enough to reach from the site of the aortic dissection in the vessel to the clinician. The approach to the aortic dissection depends on the location of the aortic dissection in the vasculature. For example, the approach can be from the femoral artery or the carotid artery.

The flexible cup 30 can be made of any flexible biocompatible material normally used for catheter balloons. For example, the flexible cup 30 can be made of polymers such as polyethylene, polyethylene terephalate (PET), nylon, polyurethane, polyether block amide (PEBAX), or the like. The suction ring 34, the ribs 37, and/or the suction ribs 38 can be fabricated as part of the flexible cup 30 or can be fabricated as separate components and attached to the body of the flexible cup. The materials for the ribs 37 can be selected from the same polymers as the flexible cup 30, such as polyethylene, polyethylene terephalate (PET), nylon, polyurethane, polyether block amide (PEBAX), or the like, or can be made of metal, such as nitinol, stainless steel, or the like. The stiffness of the flexible cup 30 can be determined as desired through selection of materials and dimensions for the flexible cup body and the ribs. For embodiments in which the flexible cup collapses toward the wall during the aortic dissection repair procedure, a flexible cup with less stiffness is desirable. For embodiments in which the flexible cup acts as a scaffold to support a sealant delivery catheter, a flexible cup with more stiffness is desirable. The shape and height of the flexible cup 30 can also be selected for a particular application as desired.

The dissection treatment system includes the dissection treatment device 20 and sealant fluid, which flows through the sealant lumen 24, into the isolation region 32, and onto and/or into the aortic dissection. In one embodiment, the sealant fluid itself passes through the sealant lumen 24, into the isolation region 32, and onto and/or into the aortic dissection. In another embodiment, a sealant delivery catheter passes through the sealant lumen 24 into the isolation region 32, and the sealant fluid passes through the sealant delivery catheter lumen onto and/or into the aortic dissection.

The sealant fluid can be any number of biodegradable or non-biodegradable polymers that can be delivered as a fluid or semi-soft solid, which hardens into a solid or cross links into a hydrogel. The fluid sealant can be a hardenable polymer that hardens or cross links with time; a two part catalyzable polymer that hardens or cross links when mixed, one of the two parts including or being a chemical catalyst; or a photoreactive catalyzable polymer that hardens when exposed to light. Examples of hardenable polymers include polyethylene glycol (PEG) polymers, polylactide (PLA) polymers, PEG polymers chemically modified with an acrylic group, PLA polymers chemically modified with an acrylic group, and the like.

Other polymers such as two part catalyzable polymers include Adherent Polymer Compositions, as described in USPGP 2005/028166 and the like. Examples of photoreactive catalyzable polymers include AdvaSeal brand polyethylene glycol-based hydrogel from the Ethicon division of Johnson & Johnson, and the like. The sealant fluid can include one or more sealant additives to induce a healing response in the aortic dissection and accelerate healing, such as fibrosing irritants, growth factors, and/or small molecules. Examples of fibrosing irritants include silk fibers, silk particles, silicon, asbestos, polyvinyl chloride (PVC), and the like. Examples of growth factors include fibroblast growth factor (FGF), vascular endothelial growth factor (VEGF), insulin-like growth factor (IGF), platelet derived growth factor (PDGF), and the like. Small molecules as defined herein include products of polymer biodegradation, proangiogenic drugs, peptides, protein growth factor, c-Jun N-terminal kinase (JNK), and the like.

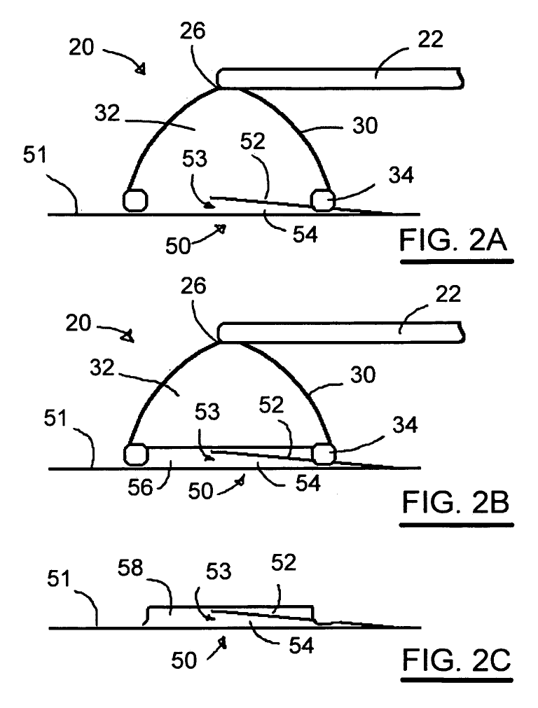

FIGS. 2A-2C are schematic cross sectional side views showing progressive steps of covering a dissection. The aortic dissection 50 in the vessel wall 51 has a false lumen 54 with an intimal flap 52. In this example, the dissection treatment device 20 forms a sealant patch over one end of the aortic dissection 50, preventing blood flow into the false lumen 54. The dissection treatment device 20 can also be used to form a sealant patch over both ends of the aortic dissection when the false lumen is a through lumen.

Referring to FIG. 2A, the aortic dissection 50 is imaged to locate the entrance 53 into the false lumen 54 of the aortic dissection 50. The dissection treatment device 20 is advanced through the vasculature in a delivery catheter (not shown) until the distal end of the delivery catheter is at the aortic dissection 50. The delivery catheter is retracted or the dissection treatment device 20 advanced so that the flexible cup 30 unfurls outside of the delivery catheter. The isolation region 32 of the flexible cup 30 using a guide catheter or balloon is located over the entrance 53 to isolate the entrance 53 from blood flow through the vessel, and the suction ring 34 placed against the vessel wall 51. The flexible cup 30 is sized so that it does not fill the vessel, so blood flow through the vessel is not blocked. Suction is applied to the suction ring 34 through the suction lumen to anchor the flexible cup 30 to the vessel wall 51. The suction ring 34 can be placed on the intimal flap 52 to hold the intimal flap 52 in position.

Referring to FIG. 2B, the sealant fluid 56 is applied to and covers the entrance 53 to the aortic dissection 50. In this example, the sealant fluid 56 is also applied within the false lumen 54. A primer, such as eosin Y or the like, can be applied to the aortic dissection 50 before application of the sealant fluid by flushing or spraying the primer through the isolation region 32. The primer increases the adhesion of the sealant patch to the vessel wall and the intimal flap.

The sealant fluid 56 can be applied to the aortic dissection 50 in a number of ways. In one method, the sealant fluid is delivered from the sealant lumen into the isolation region 32. The sealant lumen can include a sealant delivery lumen and a sealant removal lumen, so the sealant fluid enters the isolation region 32 from the sealant delivery lumen and some of the sealant fluid is withdrawn through the sealant removal lumen. More sealant fluid than desired on the aortic dissection 50 can be delivered to the isolation region 32 and a low pressure applied to the sealant lumen in the single lumen system or the sealant delivery lumen in a dual lumen system to remove excess sealant fluid. The low pressure in the isolation region 32 deforms the flexible cup 30 toward the vessel wall 51 and reduces the volume of the isolation region 32, removing the excess sealant fluid. In another method, the sealant lumen terminates in a spray head in the isolation region 32 and the sealant fluid is sprayed from the spray head, through the isolation region 32, and onto the aortic dissection 50. In yet another method, the sealant lumen can be a pair of delivery lumens and one part of the two part catalyzable polymer can be delivered to the isolation region 32 through each of the pair of delivery lumens.

Once the sealant fluid has been applied to the aortic dissection 50, the sealant fluid can be converted into a sealant patch. When the sealant fluid is a hardenable polymer or a two part catalyzable polymer, the flexible cup 30 is maintained on the aortic dissection 50 until the sealant fluid 56 hardens. When the sealant fluid is a photoreactive catalyzable polymer, the sealant fluid 56 is exposed to light until the photoreactive catalyzable polymer hardens into a sealant patch. The light in the isolation region 32 used to catalyze the polymer can be supplied by a light source disposed to provide illumination in the isolation region, such as a light source that is part of the flexible cup 30, or an external light source attached to fiber optic cable. The fiber optic cable can be permanently attached to the catheter 22 or temporarily threaded through a catheter lumen. The light is applied to the sealant fluid 56 both directly and through the intimal flap 52.

Referring to FIG. 2C, the dissection treatment device has been removed and the sealant patch 58 over the aortic dissection 50 remains. The dissection treatment device is removed by releasing the suction from the suction ring and retracting the flexible cup into the delivery catheter. Only the polymer components close to the wall where both parts of a two part polymer are present are activated by photocatalysis. The sealant patch 58 seals the entrance 53 to the false lumen 54 of the aortic dissection 50, preventing further growth of the aortic dissection due to vessel pressure and allowing the aortic dissection to heal. The sealant patch 58 can be non-biodegradable or biodegradable, depending on the selected polymer. The products of polymer biodegradation from biodegradable polymers can aid in the healing, as can sealant additives such as fibrosing irritants, growth factors, and/or small molecules. The sealant patch 58 can remain in place after the aortic dissection has healed or can be absorbed into the vessel.

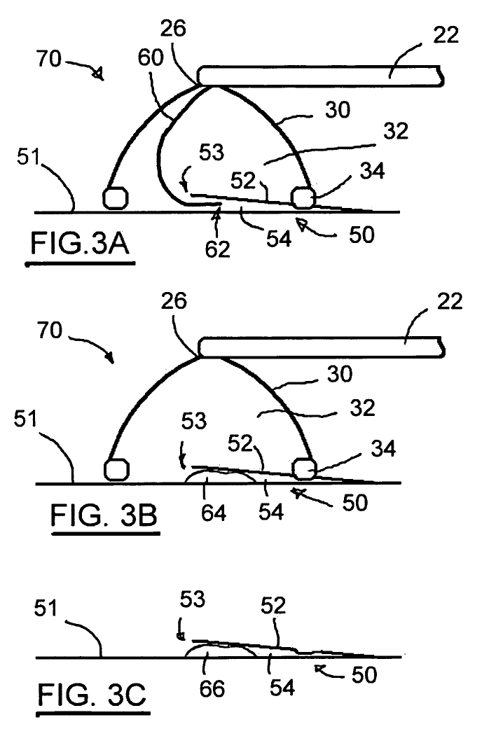

FIGS. 3A-3C are schematic cross sectional side views showing progressive steps of filling a dissection. The aortic dissection 50 in the vessel wall 51 has a false lumen 54 with an intimal flap 52. In this example, the dissection treatment device 70 forms a sealant fill within one end of the aortic dissection 50, preventing blood flow into the false lumen 54. The sealant fluid is delivered to the aortic dissection 50 with a sealant delivery catheter 60.

Referring to FIG. 3A, the aortic dissection 50 is imaged to locate the entrance 53 into the false lumen 54 of the aortic dissection 50. The dissection treatment device 70 is advanced through the vasculature in a delivery catheter (not shown) until the distal end of the delivery catheter is at the aortic dissection 50. The delivery catheter is retracted or the dissection treatment device 70 advanced so that the flexible cup 30 unfurls outside of the delivery catheter. The isolation region 32 of the flexible cup 30 is located over the entrance 53 to isolate the entrance 53 from blood flow through the vessel, and the suction ring 34 placed against the vessel wall 51. The flexible cup 30 does not fill the vessel, so blood flow through the vessel is not blocked. Suction is applied to the suction ring 34 through the suction lumen to anchor the flexible cup 30 to the vessel wall 51. The suction ring 34 can be placed on the intimal flap 52 to hold the intimal flap 52 in position.

A sealant delivery catheter 60 can be threaded through the sealant lumen of the catheter 22 into the isolation region 32 to deliver the sealant fluid to the aortic dissection 50. The sealant delivery catheter 60 can be a steerable catheter, such as the IntraLume™ Microcatheter manufactured by Medtronic, Inc. The distal tip 62 of the sealant delivery catheter 60 is advanced through the entrance 53 of the aortic dissection 50 into the false lumen 54. The distal tip 62 can be advanced to the depth within the false lumen 54 desired by the clinician and moved within the false lumen 54 while delivering the sealant fluid. A primer, such as eosin Y or the like, can be applied to the false lumen 54 with the sealant delivery catheter 60 before application of the sealant fluid. The primer increases the adhesion of the sealant fill to the vessel wall and the intimal flap. When the sealant fluid is a two part catalyzable polymer, the sealant delivery catheter lumen can be a pair of delivery lumens and one part of the two part catalyzable polymer can be delivered through each of the pair of delivery lumens. In another method, one part of the two part catalyzable polymer can be delivered through a single sealant delivery catheter lumen, then the other part of the two part catalyzable polymer delivered through the same sealant delivery catheter lumen.

Referring to FIG. 3B, the sealant delivery catheter has delivered the sealant fluid 64 and has been retracted. The sealant fluid 64 fills the entrance 53 to the aortic dissection 50. The sealant fluid can also be deposited outside the false lumen 54 when additional sealing material is desired around the entrance 53.

Once the sealant fluid has been applied to the aortic dissection 50, as for example a coating or stain such as eosin, the sealant fluid can be converted into a sealant fill. When the sealant fluid is a hardenable polymer or a two part catalyzable polymer, the flexible cup 30 is maintained on the aortic dissection 50 until the sealant fluid 64 hardens. When the sealant fluid is a photoreactive catalyzable polymer, the sealant fluid 64 is exposed to light until the photoreactive catalyzable polymer hardens into a sealant fill. The light in the isolation region 32 used to catalyze the polymer can be supplied by a light source disposed to provide illumination in the isolation region, such as a light source that is part of the flexible cup 30, or an external light source attached to fiber optic cable. The fiber optic cable can be permanently attached to the catheter 22 or temporarily threaded through a catheter lumen. The light is applied to the sealant fluid 64 through the intimal flap 52.

Referring to FIG. 3C, the dissection treatment device has been removed and the sealant fill 66 within the aortic dissection 50 remains. The dissection treatment device is removed by releasing the suction from the suction ring and retracting the flexible cup into the delivery catheter. The sealant fill 66 seals the entrance 53 to the false lumen 54 of the aortic dissection 50, preventing further growth of the aortic dissection due to vessel pressure and allowing the aortic dissection to heal. The sealant fill 66 can be non-biodegradable or biodegradable, depending on the selected polymer. The products of polymer biodegradation from biodegradable polymers can aid in the healing, as can sealant additives such as fibrosing irritants, growth factors, or small molecules. The sealant fill 66 can remain in place after the aortic dissection has healed or can be absorbed into the vessel.

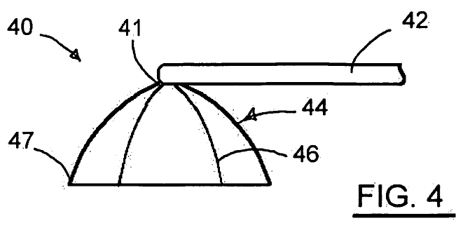

FIG. 4 is a side view of another embodiment of a dissection treatment device. The dissection treatment device 40 includes a catheter 42 and a flexible cup 44, but does not use the suction ring described in conjunction with FIGS. 1A-1C to maintain the flexible cup 44 over the aortic dissection. In the embodiment of FIG. 4, the flexible cup 44 is maintained against the vessel wall by maintaining the pressure in the isolation region within the flexible cup 44 at a lower pressure than blood pressure in the vessel. The suction lumen is in communication with the isolation region within the flexible cup 44. Ribs 46 run between the catheter attachment 41 and the circumferential edge 47. The flexibility of the flexible cup 44 can be determined by the materials, shape, and structure of the ribs 46 and flexible cup 44. The suction post is operated only when needed so that the amount of injected sealant leaving through the suction port is minimized.

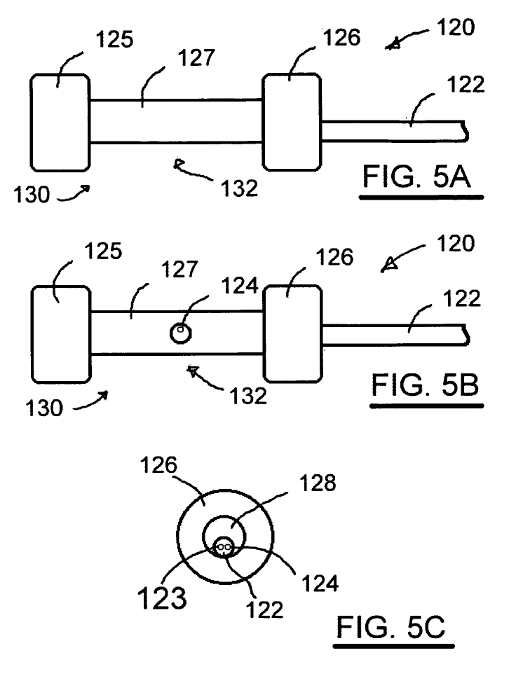

FIGS. 5A-5C are schematic side, bottom, and end views of a dissection treatment device. In this embodiment, a double balloon isolates at least a portion of an aortic dissection. A sealant fluid is delivered to the aortic dissection at the isolated portion.

The dissection treatment device 120 includes a catheter 122 and a double balloon 130. The catheter 122 defines an inflation lumen 123 and one or more sealant lumens 124 through the length of the catheter 122. The double balloon 130 includes balloons 125, 126 attached to the catheter 122, each of the balloons 125, 126 having a perfusion opening and communicating with the inflation lumen 123. A perfusion body 127 including a perfusion opening 128 connects the perfusion openings of the balloons 125, 126, so that blood can flow through the perfusion body 127 of the dissection treatment device 120 when it is deployed in a vessel. Adjoining exterior surfaces of the balloon 126, the perfusion body 127, and the balloon 125 define an isolation region 132 when the balloons 125, 126 are inflated in a vessel. The isolation region is in communication with the sealant lumen 124, so that sealant fluid is deliverable to the aortic dissection through the sealant lumen 124 and isolation region 132. In another embodiment, the balloons 125, 126 are each connected to an independent inflation lumen so the balloons can be inflated and deflated independently of each other.

The double balloon 130 is sized to fit over at least one end of the aortic dissection and the inflation lumen 123 inflates the balloons 125, 126 to maintain the double balloon 130 over the aortic dissection. The balloons 125, 126 are separated by a distance along the catheter 122, the distance being selected to accommodate at least one end of the aortic dissection in the isolation region. In one embodiment, the double balloon 130 is maintained over one end of the aortic dissection. In another embodiment, the double balloon 130 is maintained over both ends of the aortic dissection. In operation, the distal end of the dissection treatment device 120 is delivered to the aortic dissection through a delivery catheter in a rolled and/or folded configuration. The double balloon 130 unfurls to the illustrated configuration on exiting the delivery catheter. The double balloon 130 is deflated and retracted into the delivery catheter after the aortic dissection has been sealed. The double balloon 130 can optionally include light sources, fiber optic cables connected to external light sources, and/or spray heads connected the sealant lumen as desired for the selected sealant fluid and treatment method. The diameter of the perfusion body 127 can be selected to provide a desired volume in the isolation region 132.

The double balloon 130 and perfusion body 127 can be made of any flexible biocompatible material normally used for catheter balloons. For example, the double balloon 130 can be made of polymers such as polyethylene, polyethylene terephalate (PET), nylon, polyurethane, polyether block amide (PEBAX), or the like.

The catheter 122 can be made of any flexible biocompatible material normally used for catheters. For example, the catheter 122 can be made of polymers such as polyurethane, polyethylene, polyether block amide (PEBAX), nylon, composites, or any combination of the above, or the like. The catheter 122 is long enough to reach from the site of the aortic dissection in the vessel to the clinician. The approach to the aortic dissection depends on the location of the aortic dissection in the vasculature. For example, the approach can be from the femoral artery or the carotid artery.

The dissection treatment system includes the dissection treatment device 120 and sealant fluid, which flows through the sealant lumen 124, into the isolation region 132, and onto and/or into the aortic dissection. In one embodiment, the sealant fluid itself passes through the sealant lumen 124, into the isolation region 132, and onto and/or into the aortic dissection. In another embodiment, a sealant delivery catheter passes through the sealant lumen 124 into the isolation region 132, and the sealant fluid passes through the sealant delivery catheter lumen onto and/or into the aortic dissection.

The sealant fluid can be any number of biodegradable or non-biodegradable polymers that can be delivered as a fluid or semi-soft solid, which hardens into a solid or cross links into a hydrogel. The fluid sealant can be a hardenable polymer that hardens or cross links with time; a two part catalyzable polymer that hardens or cross links when mixed, one of the two parts including or being a chemical catalyst; or a photoreactive catalyzable polymer that hardens when exposed to light. Examples of hardenable polymers include polyethylene glycol (PEG) polymers, polylactide (PLA) polymers, PEG polymers chemically modified with an acrylic group, PLA polymers chemically modified with an acrylic group, and the like. Examples of two part catalyzable polymers include Adherent Polymer Compositions, as described in USPGP 2005/028166 and the like. Examples of photoreactive catalyzable polymers include AdvaSeal brand polyethylene glycol-based hydrogel from the Ethicon division of Johnson & Johnson, and the like. The sealant fluid can include one or more sealant additives to induce a healing response in the aortic dissection and accelerate healing, such as fibrosing irritants, growth factors, and/or small molecules. Examples of fibrosing irritants include silk fibers, silk particles, silicon, asbestos, polyvinyl chloride (PVC), and the like. Examples of growth factors include fibroblast growth factor (FGF), vascular endothelial growth factor (VEGF), insulin-like growth factor (IGF), platelet derived growth factor (PDGF), and the like. Small molecules as defined herein include products of polymer biodegradation, proangiogenic drugs, peptides, protein growth factor, c-Jun N-terminal kinase (JNK), and the like.

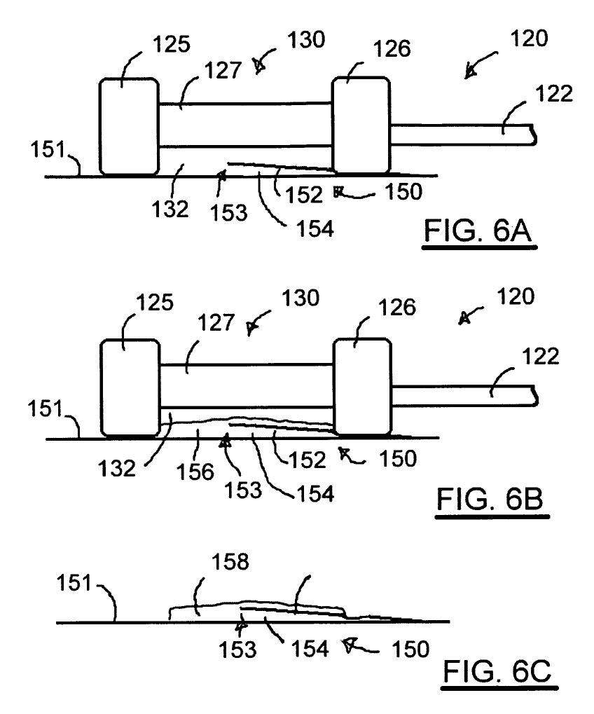

FIGS. 6A-6C are cross section side views showing progressive steps of covering a dissection. The aortic dissection 150 in the vessel wall 151 has a false lumen 154 with an intimal flap 152. In this example, the dissection treatment device 120 forms a sealant patch over one end of the aortic dissection 150, preventing blood flow into the false lumen 154. The dissection treatment device 120 can also be used to form a sealant patch over both ends of the aortic dissection when the false lumen is a through lumen.

Referring to FIG. 6A, the aortic dissection 150 is imaged to locate the entrance 153 into the false lumen 154 of the aortic dissection 150. The dissection treatment device 120 is advanced through the vasculature in a delivery catheter (not shown) until the distal end of the delivery catheter is at the aortic dissection 150. The delivery catheter is retracted or the dissection treatment device 120 advanced, so that the double balloon 130 unfurls and the balloons 125, 126 can be inflated outside of the delivery catheter. The isolation region 132 of the double balloon 130 is located over the entrance 153 to isolate the entrance 153 from blood flow through the vessel. The perfusion openings through the balloons 125, 126 and perfusion body 127 allow blood flow through the double balloon 130, so blood flow through the vessel is not blocked. The balloon 125 can be placed on the intimal flap 152 to hold the intimal flap 152 in position. Inflation pressure is applied to the balloons 125, 126 through the inflation lumen to anchor the double balloon 130 to the vessel wall 151.

Referring to FIG. 6B, the sealant fluid 156 is applied to and covers the entrance 153 to the aortic dissection 150. In this example, the sealant fluid 156 is also applied within the false lumen 154. A primer, such as eosin Y or the like, can be applied to the aortic dissection 150 before application of the sealant fluid by flushing or spraying the primer through the isolation region 132. The primer increases the adhesion of the sealant patch to the vessel wall and the intimal flap.

The sealant fluid 156 can be applied to the aortic dissection 150 in a number of ways. In one method, the sealant fluid is delivered from the sealant lumen into the isolation region 132. The sealant lumen can include a sealant delivery lumen and a sealant removal lumen, so the sealant fluid enters the isolation region 132 from the sealant delivery lumen and some of the sealant fluid is withdrawn through the sealant removal lumen. More sealant fluid than desired on the aortic dissection 150 can be delivered to the isolation region 132 and a low pressure applied to the sealant lumen in the single lumen system or the sealant delivery lumen in a dual lumen system to remove excess sealant fluid. The low pressure in the isolation region 132 deforms the double balloon 130 toward the vessel wall 151 and reduces the volume of the isolation region 132, removing the excess sealant fluid. In another method, the sealant lumen terminates in a spray head in the isolation region 132 and the sealant fluid is sprayed from the spray head, through the isolation region 132, and onto the aortic dissection 150. In yet another method, the sealant lumen can be a pair of delivery lumens and one part of the two part catalyzable polymer can be delivered to the isolation region 132 through each of the pair of delivery lumens.

Once the sealant fluid has been applied to the aortic dissection 150, the sealant fluid can be converted into a sealant patch. When the sealant fluid is a hardenable polymer or a two part catalyzable polymer, the double balloon 130 is maintained on the aortic dissection 150 until the sealant fluid 156 hardens. When the sealant fluid is a photoreactive catalyzable polymer, the sealant fluid 156 is exposed to light until the photoreactive catalyzable polymer hardens into a sealant patch. The light in the isolation region 132 used to catalyze the polymer can be supplied by a light source disposed to provide illumination in the isolation region, such as a light source that is part of the double balloon 130, or an external light source attached to fiber optic cable. The fiber optic cable can be permanently attached to the catheter 122 or temporarily threaded through a catheter lumen. The light is applied to the sealant fluid 156 both directly and through the intimal flap 152.

Referring to FIG. 6C, the dissection treatment device has been removed and the sealant patch 158 over the aortic dissection 150 remains (a thin layer of polymer at the vessel wall where the two part polymer has reacted (catalyzed) to form a stable layer). The dissection treatment device is removed by deflating the double balloons and retracting the double balloons into the delivery catheter. The sealant patch 158 seals the entrance 153 to the false lumen 154 of the aortic dissection 150, preventing further growth of the aortic dissection due to vessel pressure and allowing the aortic dissection to heal. The sealant patch 158 can be non-biodegradable or biodegradable, depending on the selected polymer. The products of polymer biodegradation from biodegradable polymers can aid in the healing, as can sealant additives such as fibrosing irritants, growth factors, and/or small molecules. The sealant patch 158 can remain in place after the aortic dissection has healed or can be absorbed into the vessel.

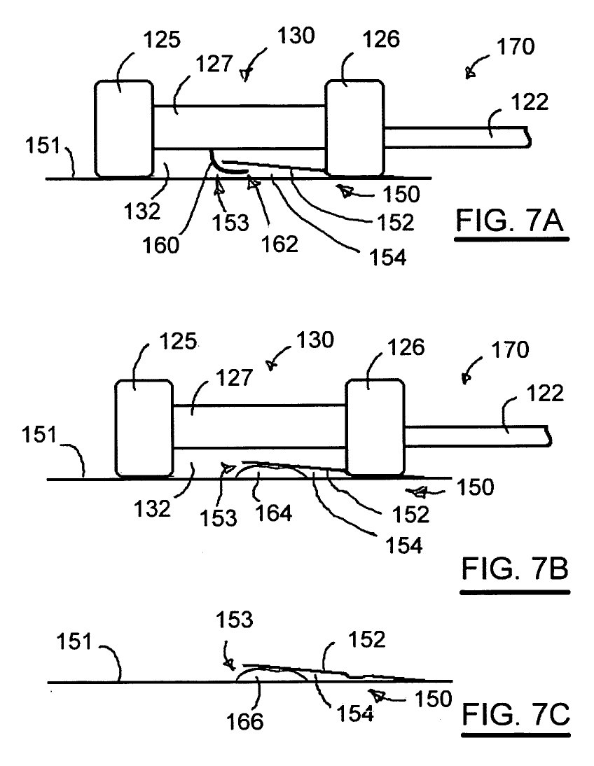

FIGS. 7A-7C are schematic cross sectional side views showing progressive steps of filling a dissection. The aortic dissection 150 in the vessel wall 151 has a false lumen 154 with an intimal flap 152. In this example, the dissection treatment device 120 forms a sealant fill within one end of the aortic dissection 150, preventing blood flow into the false lumen 154. The sealant fluid is delivered to the aortic dissection 150 with a sealant delivery catheter 160.

Referring to FIG. 7A, the aortic dissection 150 is imaged to locate the entrance 153 into the false lumen 154 of the aortic dissection 150. The dissection treatment device 170 is advanced through the vasculature in a delivery catheter (not shown) until the distal end of the delivery catheter is at the aortic dissection 150. The delivery catheter is retracted or the dissection treatment device 170 advanced, so that the double balloon 130 unfurls and the balloons 125, 126 can be inflated outside of the delivery catheter. The isolation region 132 of the double balloon 130 is located over the entrance 153 to isolate the entrance 153 from blood flow through the vessel. The perfusion openings through the balloons 125, 126 and perfusion body 127 allow blood flow through the double balloon 130, so blood flow through the vessel is not blocked. The balloon 125 can be placed on the intimal flap 152 to hold the intimal flap 152 in position. Inflation pressure is applied to the balloons 125, 126 through the inflation lumen to anchor the double balloon 130 to the vessel wall 151.

A sealant delivery catheter 160 can be threaded through the sealant lumen of the catheter 122 into the isolation region 132 to deliver the sealant fluid to the aortic dissection 150. The sealant delivery catheter 160 can be a steerable catheter, such as the IntraLume™ Microcatheter manufactured by Medtronic, Inc. The distal tip 162 of the sealant delivery catheter 160 is advanced through the entrance 153 of the aortic dissection 150 into the false lumen 154. The distal tip 162 can be advanced to the depth within the false lumen 154 desired by the clinician and moved within the false lumen 154 while delivering the sealant fluid. A primer, such as eosin Y or the like, can be applied to the false lumen 154 with the sealant delivery catheter 160 before application of the sealant fluid. The primer increases the adhesion of the sealant fill to the vessel wall and the intimal flap. When the sealant fluid is a two part catalyzable polymer, the sealant delivery catheter lumen can be a pair of delivery lumens and one part of the two part catalyzable polymer can be delivered through each of the pair of delivery lumens. In another method, one part of the two part catalyzable polymer can be delivered through a single sealant delivery catheter lumen, then the other part of the two part catalyzable polymer delivered through the same sealant delivery catheter lumen.

Referring to FIG. 7B, the sealant delivery catheter has delivered the sealant fluid 164 and has been retracted. The sealant fluid 164 fills the entrance 153 to the aortic dissection 150. The sealant fluid can also be deposited outside the false lumen 154 when additional sealing material is desired around the entrance 153.

Once the sealant fluid has been applied to the aortic dissection 150, the sealant fluid can be converted into a sealant fill. When the sealant fluid is a hardenable polymer or a two part catalyzable polymer, the double balloon 130 is maintained on the aortic dissection 150 until the sealant fluid 164 hardens. When the sealant fluid is a photoreactive catalyzable polymer, the sealant fluid 164 is exposed to light until the photoreactive catalyzable polymer hardens into a sealant fill. The light in the isolation region 132 used to catalyze the polymer can be supplied by a light source disposed to provide illumination in the isolation region, such as a light source that is part of the double balloon 130, or an external light source attached to fiber optic cable. The fiber optic cable can be permanently attached to the catheter 122 or temporarily threaded through a catheter lumen. The light is applied to the sealant fluid 164 through the intimal flap 152.

Referring to FIG. 7C, the dissection treatment device has been removed and the sealant fill 166 within the aortic dissection 150 remains. The dissection treatment device is removed by releasing pressure from the balloons and retracting the double balloon into the delivery catheter. The sealant fill 166 seals the entrance 153 to the false lumen 154 of the aortic dissection 150, preventing further growth of the aortic dissection due to vessel pressure and allowing the aortic dissection to heal. The sealant fill 166 can be non-biodegradable or biodegradable, depending on the selected polymer. The products of polymer biodegradation from biodegradable polymers can aid in the healing, as can sealant additives such as fibrosing irritants, growth factors, or small molecules. The sealant fill 166 can remain in place after the aortic dissection has healed or can be absorbed into the vessel.

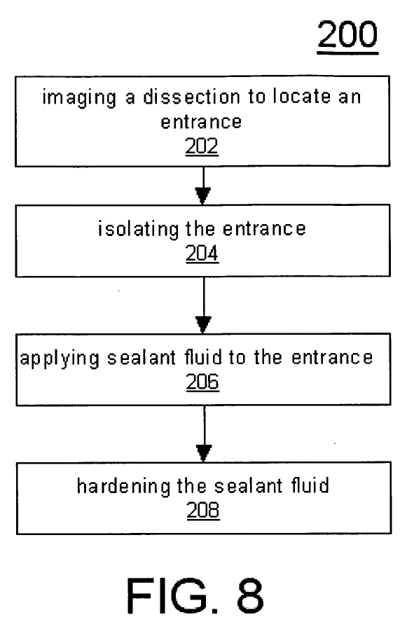

FIG. 8 is a flowchart of a method of aortic dissection treatment. The method 200 includes the steps of imaging the aortic dissection to locate an entrance (202); isolating the entrance (204) from blood flow through the vessel without blocking the blood flow through the vessel; and applying sealant fluid to the entrance (206). The method 200 can further include the step of hardening the sealant fluid (208).

The step of imaging the aortic dissection to locate an entrance (202) can be performed with any imaging system, such as an intravascular ultrasound (IVUS) system. Isolating the entrance (204) from blood flow through the vessel without blocking the blood flow through the vessel can be performed with an aortic dissection treatment system having a flexible cup or a double balloon as described above. Applying sealant fluid to the entrance (206) can include applying sealant fluid within a false lumen of the aortic dissection and/or applying sealant fluid over an intimal flap of the aortic dissection. The sealant fluid can include an additive for irritating the aortic dissection to promote healing. A primer can be applied to the aortic dissection before applying the sealant fluid to initiate a photoreaction and/or to increase adhesion of the sealant fluid to the aortic dissection. Hardening the sealant fluid (208) can include hardening the sealant fluid by exposing the sealant fluid to light or hardening the sealant fluid with a chemical catalyst.

While specific embodiments according to the invention are disclosed herein, various changes and modifications can be made without departing from its spirit and scope.

1. A treatment system for an aortic dissection comprising:
a catheter defining a suction lumen and a sealant lumen;
a flexible cup defining an isolation region within the flexible cup, the isolation region being in communication with the suction lumen and the sealant lumen; and
a sealant fluid deliverable to the aortic dissection through the sealant lumen and the isolation region;
wherein the flexible cup is sized to fit over at least one end of the aortic dissection and the suction lumen applies suction to maintain the flexible cup over the at least one end of the aortic dissection.

2. The treatment system of claim 1 further comprising a suction ring around a circumferential edge of the flexible cup, the suction ring defining a plurality of suction ports and being in communication with the suction lumen.

3. The treatment system of claim 2 wherein the suction ring is in communication with the suction lumen through a suction rib.

4. The treatment system of claim 1 further comprising ribs disposed around the flexible cup between the catheter attachment and the circumferential edge of the flexible cup.

5. The treatment system of claim 1 wherein the sealant fluid is a hardenable polymer.

6. The treatment system of claim 1 wherein the sealant fluid is a two part catalyzable polymer.

7. The treatment system of claim 1 wherein the sealant fluid is a photoreactive catalyzable polymer.

8. The treatment system of claim 1 wherein the sealant fluid includes fibrosing irritants.

9. The treatment system of claim 1 wherein the sealant fluid includes growth factors.

10. The treatment system of claim 1 wherein the sealant fluid includes small molecules.

11. The treatment system of claim 1 further comprising a primer deliverable to the aortic dissection through the sealant lumen and the isolation region.

12. The treatment system of claim 1 further comprising a sealant delivery catheter slidable through the sealant lumen, the sealant fluid being deliverable to the aortic dissection through the sealant delivery catheter.

13. The treatment system of claim 1 further comprising a light source disposed to provide illumination in the isolation region.

14. A treatment system for an aortic dissection in a vessel comprising:
a catheter defining an inflation lumen and a sealant lumen;
a first balloon attached to the catheter, the first balloon defining a first perfusion opening and being in communication with the inflation lumen;
a second balloon attached to the catheter, the second balloon defining a second perfusion opening and being in communication with the inflation lumen;
a perfusion body connecting the first perfusion opening and the second perfusion opening, with adjoining exterior surfaces of the first balloon, the second balloon, and the perfusion body defining an isolation region when the first balloon and the second balloon are inflated in the vessel, the isolation region being in communication with the sealant lumen; and
a sealant fluid deliverable to the aortic dissection through the sealant lumen and isolation region;
wherein the first balloon and the second balloon are separated by a distance along the catheter, the distance being selected to accommodate at least one end of the aortic dissection in the isolation region.

15. The treatment system of claim 13 wherein the sealant fluid is a hardenable polymer.

16. The treatment system of claim 13 wherein the sealant fluid is a two part catalyzable polymer.

17. The treatment system of claim 13 wherein the sealant fluid is a photoreactive catalyzable polymer.

18. The treatment system of claim 13 wherein the sealant fluid includes fibrosing irritants.

19. The treatment system of claim 13 wherein the sealant fluid includes growth factors.

20. The treatment system of claim 13 wherein the sealant fluid includes small molecules.

21. The treatment system of claim 13 further comprising a primer deliverable to the aortic dissection through the sealant lumen and the isolation region.

22. The treatment system of claim 13 further comprising a sealant delivery catheter slidable through the sealant lumen, the sealant fluid being deliverable to the aortic dissection through the sealant delivery catheter.

23. The treatment system of claim 13 further comprising a light source disposed to provide illumination in the isolation region.

24. A method of treatment for an aortic dissection in a vessel, the method comprising:
imaging the aortic dissection to locate an entrance;
isolating the entrance from blood flow through the vessel without blocking the blood flow through the vessel; and
applying sealant fluid to the entrance.

25. The method of claim 23 wherein the isolating the entrance comprises isolating the entrance within a flexible cup.

26. The method of claim 23 wherein the isolating the entrance comprises isolating the entrance within a double balloon.

27. The method of claim 23 wherein the applying sealant fluid comprises applying sealant fluid within a false lumen of the aortic dissection.

28. The method of claim 23 wherein the applying sealant fluid comprises applying sealant fluid over an intimal flap of the aortic dissection.

29. The method of claim 23 further comprising hardening the sealant fluid.

30. The method of claim 28 wherein the hardening comprises hardening the sealant fluid by exposing the sealant fluid to light.

31. The method of claim 28 wherein the hardening comprises hardening the sealant fluid with a chemical catalyst.

32. The method of claim 23 further comprising priming the aortic dissection before the applying sealant fluid.

33. The method of claim 23 wherein the sealant fluid includes an additive for irritating the aortic dissection to promote healing.
